(19) United States
(12) Patent Application Publication
(10) Pub. No.: US 2005/0163718 A1
(43) Pub. Date: Jul. 28, 2005

(54) Injectable euthanasia compositions that include a taste aversive agent

(76) Inventor: Jack I. Shugart, Parkville, MO (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 10/764,222

(22) Filed: Jan. 23, 2004

(51) Int. Cl. A01N 43/42; A61K 9/44

(52) U.S. Cl. 424/10.2

(57) ABSTRACT

An injectable euthanasia composition for animals is provided. This composition includes a euthanasia formulation and a taste aversive agent. The taste aversive agent is included in a concentration sufficient to induce an aversive response in humans or other animals if the euthanasia composition is ingested or inhaled. Although the potential for intentional ingestion or inhalation is greatly reduced by the addition of the taste aversive agent, the effectiveness of the euthanasia composition to euthanize animals is not diminished.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

The present invention relates to injectable euthanasia compositions for use in veterinary applications and to methods of making and using these compositions. More specifically, the present invention relates to injectable euthanasia compositions that include a taste aversive agent that does not diminish the effectiveness of the euthanasia composition when administered by injection.

A disadvantage of currently available injectable euthanasia compositions is the potential for drug abuse through intentional ingestion or inhalation. Most currently available euthanasia formulations include barbiturates or other government controlled drugs with potential for abuse of active agents. Thus, there is much concern for safety and misuse in situations where it is desirable to have euthanasia formulations readily available, such as for veterinary use.

Previous efforts to address concerns for safety and misuse include restricting access to the compositions, labeling, and/or adding a noticeable colorant to the composition to serve as a reminder of the need for proper storage. One problem with labeling and use of colorants is that the labeling and/or colorant may be overlooked, and therefore, these measures may be ineffective to encourage proper storage. Another disadvantage with labeling or addition of colorant is that it is not effective to discourage intentional ingestion or inhalation and may actually invite abuse.

An additional disadvantage with currently available injectable euthanasia formulations may arise when euthanasia is performed in the field, such as with the euthanizing of horses and other farm animals. If the carcass of a euthanized animal is not removed, domesticated animals and wildlife may be poisoned by ingesting portions of the injected carcass. Furthermore, if euthanizing agents are taken into the field in unit dose forms, such as syringes, the composition may be more easily accessible to unauthorized users.

In order to overcome these disadvantages, improved injectable euthanasia compositions that discourage intentional ingestion and/or inhalation, without diminishing effectiveness, are needed.

### BRIEF SUMMARY OF THE INVENTION

It is an object of the present invention to provide an injectable euthanasia composition that includes a taste aversive agent so that the risk of accidental or intentional ingestion and inhalation is greatly reduced.

It is another object of the present invention to provide an injectable euthanasia composition that includes a taste aversive agent and yet retains its effectiveness when injected into an animal.

According to the present invention, the foregoing and other objects are achieved by an injectable euthanasia composition that includes a mixture of an injectable euthanasia formulation and a taste aversive agent. This composition is made by combining the components together to form a mixture. The euthanasia composition of the present invention may be injected intravenously, intraperitoneally, intrathoracically, intracardially, or by other nonvascular routes. This composition may be administered by injection either with or without premedication such as an anesthetic, an analgesic and/or a tranquilizer.

Additional objects, advantages and novel features of the present invention will be set forth in part in the description which follows, and in part will become apparent to those skilled in the art upon examination of the following, or may be learned from the practice of the invention. The objects and advantages of the invention may be realized and attained by means of the instrumentalities and the combinations particularly pointed out in the appended claims.

### DETAILED DESCRIPTION OF PREFERRED EMBODIMENT

The present invention is an injectable euthanasia composition. This composition includes, but is not limited to, a mixture of a euthanasia formulation and a taste aversive agent. It functions to provide a safety measure to deter ingestion and inhalation by humans or other animals, without significantly diminishing the effectiveness of the euthanasia formulation when it is administered by injection to animals.

In order to more fully understand the present invention, the following definitions are provided:

“Taste aversive agent” refers to a substance that is intended to induce an aversive response in humans or other animals when ingested or inhaled and, thereby, deters further ingestion or inhalation.

“Denatonium benzoate” refers to benzyldiethyl[(6-xylylcarbamoyl)methyl] ammonium benzoate and includes any taste aversive denatonium salts.

“Euthanasia formulation” refers to those agents known in the art to produce euthanasia and includes such agents alone or mixtures of such agent(s) with one or more other substances, such as cardiotoxic agents, tranquilizers, and other substances recognized in the art to enhance various properties of the euthanasia agent.

The taste aversive agent used in the composition of the present invention can be, but is not limited to, a bittering agent. Preferably, denatonium benzoate is used as the taste aversive agent. However, those skilled in the art will recognize that alternative taste aversive agents may be incorporated into the composition of the present invention to achieve the same objects.

The amount of the taste aversive agent to be included in the composition of the present invention is an amount that is effective, when combined with the euthanasia formulation, to deter further ingestion or inhalation, without significantly diminishing the effectiveness of the injectable euthanasia composition. When denatonium benzoate is used as the taste aversive agent, it is included at about 2 to about 1000 ppm by volume of the injectable euthanasia composition of the present invention. Preferably, the composition includes about 5 to about 500 ppm by volume denatonium benzoate. More preferably, the composition includes about 50 to about 200 ppm by volume denatonium benzoate. Most preferably, the composition includes about 90 to about 110 ppm by volume denatonium benzoate.

Denatonium benzoate has previously been included in household products, personal care products, pesticides, and automotive products to prevent accidental ingestion. Such products are not intended to be injected or ingested. However, there are no known reports of the use of denatonium benzoate, or other taste aversive agents, in a euthanasia composition intended to be administered by injection.

The injectable euthanasia formulation used in the composition of the present invention may include barbituric acid derivatives such as pentobarbital, amobarbital, and/or hexobarbital. Examples of suitable euthanasia formulations are described in U.S. Pat. Nos. 5,962,536; 5,281,611; and 5,290,775; and U.S. Patent Application Pub. No. 20030159659. Preferable formulations are those described in U.S. Pat. Nos. 5,281,611 and 5,290,775. Each patent and patent application cited herein is incorporated by reference in its entirety. The choice of euthanasia formulation may depend, for example, upon the specific application, ease of use, and associated side effects.

More preferably, the formulation of the present invention includes, in admixture, an injectable aqueous solution of a cardiotoxic compound such as a quinacrine salt or a chloroquine salt and gamma-hydroxybutramide (embutramide) (CAS No. 15687-14-6). Most preferably, the formulation also includes lidocaine. The lidocaine may be water solubilized lidocaine or water soluble salts of lidocaine. The formulation may but need not include a water soluble inorganic salt such as an alkali metal salt and/or an alkaline earth metal salt other than a sodium salt. Preferably, the formulation does not contain a water soluble inorganic salt. The preferred composition includes as solids alone between about 70 and 80 percent by weight of the gamma-hydroxybutramide, about 15 to 25 percent by weight of the chloroquine compound, and about 0.5 to 2.0 percent of the lidocaine compound. The composition preferably contains as liquids a mixture of about 40 percent by weight water and about 60 percent by weight solvent. Preferably, the solvent is ethanol and/or denatured alcohol and is present in an amount sufficient to dissolve the solids. The solids are preferably between about 20 and 30 percent by weight of the solution. Within these ranges, the lethal dosage of the composition as an aqueous solution injected into a dog or a cat is preferably between about 0.10 and 0.50 ml per kg of body weight. More preferably, it may include gamma-hydroxybutramide and a chloroquine compound or quinacrine compound in a ratio of between about 3 to 1 and 6 to 1, and a ratio of lidocaine to gamma-hydroxybutramide of between about 0.01 and 0.015 to 1, with the ingredients being included in an amount sufficient to produce euthanasia. Most preferably, it may include chloroquine diphosphate or chloroquine base or quinacrine hydrochloride or quinacrine base.

While the taste aversive agent and an injectable euthanasia formulation are the only components necessary in the composition of the present invention, a number of optional components may be added to enhance various properties of the formulation. Such optional components may include, but are not limited to, a colorant, a buffer, a solubilizing agent, or combinations thereof.

The injectable euthanasia composition of the present invention is made by combining a taste aversive agent with an injectable euthanasia formulation to form a mixture. Preferably, denatonium benzoate is used as the taste aversive agent. If denatonium benzoate is used as the taste aversive agent, it may be solubilized prior to mixing it with the euthanasia formulation. Alternatively, it may be added directly to the formulation and mixed therewith until it is dissolved. One preferred method of making the composition of the present invention is described in Example 1.

The euthanasia composition of the present invention is to be administered as an injectable composition to an animal. It may be injected intravenously, intraperitoneally, intrathoracically, intracardially, or by other nonvascular routes. Preferably, it is administered by intravenous injection using a syringe and needle appropriate to route of injection and species. Preferably, the euthanasia composition is administered for about 5-30 seconds. Most preferably, the euthanasia composition is administered for about 10-15 seconds. Preferably, the composition of the present invention is administered to cats, dogs, horses, lab animals such as rats and mice, and other mammals in need thereof. There is no difference in pain at the injection site between a euthanasia formulation with a taste aversive agent added thereto and a euthanasia formulation without a taste aversive agent.

The dosage of the composition administered should be based on the particular euthanasia formulation included in the composition of the present invention. The addition of the taste aversive agent does not significantly diminish the effectiveness of the euthanasia formulation, and therefore, its addition should not alter the dosage administered.

Death of the treated animal may be determined by cessation of brain waves, respiration or other vital signs. The time for the euthanasia formulation to cause cessation of brain waves is not altered by the addition of the taste aversive agent.

The composition may, but need not, be administered with premedication. Premedication may help to avoid discomfort or unwanted side effects in the animal. If premedication is used, anesthetics, tranquilizers and/or analgesics may be used. Preferably, an anesthetic such as propofol is administered by slow intravenous infusion a few seconds to no more than a minute prior to injection of the euthanasia composition with additional give or take thereafter as necessary to establish a general plane of anesthesia. Most preferably, the anesthetic is administered about 10-60 seconds prior to injection of the euthanasia composition. If an analgesic and/or tranquilizer are included as part of the premedication, then, typically, they will be administered in combination shortly before the anesthetic is administered.

The following are examples of compositions and methods of using these compositions that are within the scope of the present invention. These examples are offered by way of illustration and are not intended to limit the scope of the invention in any manner.

### EXAMPLE 1

### Preparation of Injectable Euthanasia Composition

100.0 Kg of purified water, USP were added to a 1000 L tank. Agitation began. 7.5 g FD&C Green #3 were added to the water. 11.6 Kg of chloroquine phosphate, USP were added, and agitation was continued until the chloroquine phosphate dissolved. 92.5 Kg of ethyl alcohol, 190 proof, USP were added. Agitation was continued until the alcohol dissolved. Next, 35.0 Kg of gamma-hydroxybutramide were added, and agitation continued until it dissolved. With continued agitation, 500.0 g of lidocaine, USP were added to the solution, and it was agitated until the lidocaine dissolved. Approximately 500 mL of the solution were removed. 25.0 g of denatonium benzoate were slowly but immediately added to the 500 mL solution removed from the batch. The 500 mL of denatonium benzoate solution were then added to the batch. The container that held the removed solution was then rinsed with additional aliquots from the batch. The solution was then brought to a weight of 250 Kg by adding 10.4 Kg of purified water, USP (this being the Q.S. amount). The solution was then agitated for 15 minutes and filtered through a 10 micron cartridge filter.

### EXAMPLE 2

### Method for Providing Euthanasia in an Animal

The injectable euthanasia composition of Example 1 was injected into an animal intravenously to achieve euthanasia in the animal. Death was determined by measuring vital signs.

### EXAMPLE 3

### Method of Providing Euthanasia Including Premedication with an Anesthetic

The method of Example 2 was preceded by intravenous premedication with 5.5 mg/Kg of propofol at 51 seconds prior to injection of the euthanasia composition of Example 1.

From the foregoing, it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and sub-combinations are of utility and may be employed without reference to other features and sub-combinations. This is contemplated by and is within the scope of the claims.

1. An injectable euthanasia composition, comprising the mixture of:
a euthanasia formulation in an amount sufficient to produce euthanasia; and
a taste aversive agent,
wherein said taste aversive agent is provided in a concentration sufficient to render said composition aversive to an animal when ingested or inhaled.

2. The injectable euthanasia composition of claim 1 wherein said animal is a person.

3. The euthanasia composition of claim 1 wherein said taste aversive agent comprises denatonium benzoate.

4. The euthanasia composition of claim 3 wherein said denatonium benzoate is about 2 to about 1000 ppm by volume of said composition.

5. The euthanasia composition of claim 3 wherein said denatonium benzoate is about 5 to about 500 ppm by volume of said composition.

6. The euthanasia composition of claim 3 wherein said denatonium benzoate is about 50 to about 200 ppm by volume of said composition.

7. The euthanasia composition of claim 1 wherein said taste aversive agent tastes bitter.

8. The euthanasia composition of claim 1 wherein said euthanasia formulation comprises in admixture in an injectable aqueous solution:
a cardiotoxic compound selected from the group consisting of a quinacrine compound, a chloroquine compound, salts thereof, and combinations thereof; and
gamma-hydroxybutramide,
wherein said solution contains a ratio of said gamma-hydroxybutramide to said cardiotoxic compound of between about 3 to 1 and about 6 to 1.

9. The euthanasia composition of claim 8, further comprising:
a lidocaine selected from the group consisting of a water solubilized lidocaine and water soluble salts thereof, wherein said solution contains a ratio of lidocaine to gamma-hydroxybutramide of between about 0.01 and 0.015 to 1.

10. The euthanasia composition of claim 9 wherein said chloroquine compound is chloroquine diphosphate or chloroquine base and said quinacrine compound is quinacrine hydrochloride or quinacrine base.

11. The euthanasia composition of claim 1 wherein said euthanasia formulation comprises in admixture in an aqueous solution:
gamma-hydroxybutramide dissolved in a water immiscible liquid solubilizing agent;
a water soluble chloroquine compound; and
a lidocaine compound,
wherein said solution contains a ratio of gamma-hydroxybutramide to chloroquine salt of between about 3 to 1 and 6 to 1 and a radio of lidocaine compound to gamma-hydroxybutramide between about 0.01 to 1 and about 0.015 to 1.

12. The euthanasia composition of claim 11 wherein said solubilizing agent is selected from the group consisting of ethanol, denatured alcohol, and combinations thereof and the chloroquine compound is chloroquine diphosphate or chloroquine base.

13. A method for euthanizing an animal, comprising:
providing a euthanasia composition comprising the mixture of a euthanasia formulation and a taste aversive agent; and
injecting said euthanasia composition into said animal,
wherein euthanasia occurs in the animal.

14. The method of claim 13 wherein said taste aversive agent comprises denatonium benzoate.

15. The method of claim 13, further comprising:
premedicating said animal with an anesthetic prior to injecting said euthanasia composition into said animal.

16. The method of claim 15 wherein said anesthetic is propofol.

17. The method of claim 13, further comprising:
premedicating said animal with a tranquilizer prior to injecting said euthanasia composition into said animal.

18. The method of claim 17 wherein said animal is further premedicated with an analgesic.

19. A method for making a taste aversive injectable euthanasia composition, comprising:
mixing a taste aversive agent with a sufficient amount of a euthanasia formulation to produce euthanasia.
